(19) United States
(12) Patent Application Publication
(10) Pub. No.: US 2016/0366017 A1
(43) Pub. Date: Dec. 15, 2016

(54) Method of network traffic management in information and communication systems

(71) Applicant: Allnet Broker Sp. Zo. o., Leszno (PL)

(72) Inventor: Krzysztof Graczykowski, Wroclaw (PL)

(73) Assignee: ALLNET BROKER SP. Z O. O., Leszno (PL)

(21) Appl. No.: 14/773,408

(22) PCT Filed: Jun. 9, 2015
(86) PCT No.: PCT/PL2015/000090

§ 371 Date: Sep. 8, 2015

(51) Int. Cl. H04L 12/24; H04L 12/26; H04L 29/08

(52) U.S. Cl. CPC H04L 41/0816; H04L 41/5003; H04L 67/22; H04L 43/08; H04L 41/0823

(57) ABSTRACT

The subject of the invention is a method of network traffic management in information and communication systems (in particular, in active network devices) where it is desired/operated/insufficient to use the QoS (Quality of Service) characteristics in order to improve the quality of information and communication services. The method lies in the fact that, by using a multilayer neural network, knowledge base and feedback, the system is able to autonomously adjust QoS parameters and settings in the device to the existing conditions and circumstances.

The subject of the invention is a method of network traffic (sent data packets, established connections) management (selection of rules, priorities and other parameters) in information and communication systems (information and communication networks, in particular, in active mediation devices in information and communication networks). It is most suitable to use the invention in embedded systems (/modules) of devices for network traffic management. The invention is particularly advantageous in systems where it is desired/operated/insufficient to use the QoS (Quality of Service) characteristics in order to improve the quality of information and communication services.

The available in the market elements of information and communication systems for network traffic management (most of which are managed switches and routers) come with various solutions aimed at enabling the user to keep negative connection parameters at the lowest level through proper configuration of some operating parameters of the device. Examples of the above negative parameters, among other things, are; network/link congestion (even a single computer can use/take 100% bandwidth of a wireless network access point; some services/servers may require multiple requests/connections; P2P networks can significantly load the network due to their good scalability; DDoS attacks also constitute a considerable risk in this regard), latency (significant and uneven delay of individual packets waiting time between action taken by the user of the information system and reaction of the system, resulting from the communication time delays), packet loss, jitter (level of diversification of latency for individual packets, particularly important in VoIP) and other. The most popular method of preventing network congestion, currently used in routers and other manageable active network devices, is bandwidth throttling (by defining limits for user/device/session/connection); however, manufacturers of network equipment can partially cope with the latency and jitter by introducing prioritization of services which are more important and sensitive to these parameters.

The most common solution/approach used to enable efficient configuration of device which automatically manages network traffic is to implement in it functionalities described in the document ITU-T E.800 (ITU-T Recommendations, ITU-T E800: Definitions of terms related to quality of service, latest version (September 2008), resources available online, access 2015-0345, http://www.itu.int/ITU-T/recommendations/rec.aspx?rec=9524) T/recommendations/rec.aspx?rec9524) (and subsequent), The document is an ITU (International Telecommunication Union) recommendation related to the quality of telecommunication services, and, more precisely, the so-called QoS (Quality of Service). QoS is available to the vast majority of contemporary manageable network devices, but in a very different range. In some devices, it can only enable/disable QoS and the user has no insight into the principle and scope of QoS operation on the device, and in others, there is much more options (CISCO, Cisco IOS Quality of Service Solutions Configuration Guide, Release 12.2, resources available online, access on 2015-03-22, http://www.cisco.com/c/en/us/td/docs/ios/12_2/qos/configuration/guide/fqos_c.ht). All devices offering QoS must (ITU-T Recommendations) meet the assumptions of that specification in order to allow for communication between devices provided by different manufacturers while maintaining a coherent strategy of network traffic management, and similar behaviour of devices from different manufacturers while changing their corresponding options/parameters. All currently produced network devices typically operate in one of the following three modes: standard (network traffic management using basic settings, set ‘rigidly’ in the configuration panel of the device, on Its internal website), standard+QoS (functionality of QoS mode of the device is described in ITU-T (ITU-T Recommendations), is used to a varying extent, but usually allows the user to: prioritize services, prioritize packets by size, prioritize ICMP packets, throttle bandwidth, bandwidth rule, prevent congestion, protect against flood type attacks) and QoS mode which additionally takes into account management policies. The method according to the invention is most similar to the second mode described above, i.e. QoS.

There is a vast number of papers considering theoretical possibility of using artificial neural networks (ANN) for network traffic modelling, simulation and control. In 1997, there was published an article considering the possibility of forecasting network traffic related to the streaming of image in MPEG in the ATM networks (Lagkas T., Angelidis P., Georgiadis L., Wireless Network Traffic and Quality of Service Support Trends and Standards, pages 89-91, Information Science Reference, IGI Global, Hershey, N.Y., 2010), Only after the first implementation successes of hierarchical ANN in 2007, after 2014, scientific articles on the application of ANN for network traffic modelling began to appear, which stated, apart from theory, the results of experiments/measurements (for instance, Gao Q., Li G., A Traffic Prediction Method based on ANN and Adaptive Template Matching, Przeglad Elektrotechniczny, ISSN 0033-2097, November 2011; Haviluddin H., Rayner A., Daily Network Traffic Prediction Based on Backpropagation Neural Network, Australian Journal of Basic and Applied Sciences, December 2014); however, the measurements were usually performed on a dedicated computer/server with two network cards, acting as a mediation device for the transmission of data (inserted in the data path). However, ANN have not been used for network traffic management so far (there are theoretical solutions and prototype/research implementations on modelling, but solutions using ANN for management are not available in the market).

The essence of the method according to the invention is that, in addition to the ability to respond to common events foreseen by the administrator/manufacturer in the policies, it is able to respond to other, unexpected events and situations, since, in addition to a configurable set of policies, it has an implemented multilayer artificial neural network which analyzes in real time, in discrete points in time, the current parameters of individual connections, the current parameters of active data transmission though the device, and the current settings of the device. Based on simple input data comprising, among other things: above data (observations) and the collected data relating to the current activity of users and devices in the network, and archival data concerning their activity, gathered in the internal knowledge base (knowledge), the aforementioned neutral network is able to autonomously and unsupervisedly define and set new values of parameters and settings for Qos (QoS settings) on a managed network device. Previous parameters and settings are changed without reset, thus allowing trouble-free operation despite the changes of parameters and settings, wherein the user is also able to affect (configuration) the strategy of QoS parameters and settings selection via the intranet site or file with configuration of autonomous mode settings (mode settings). Autonomous mode settings, i.e. mode using the invention, are realised in a clear and comprehensive way for users using linguistic variables with values in any min-max range, such as: “reliability”, “security” or “efficiency”. The values of these variables affect each other which means that for example if the user increases the value of “efficiency” variable, the system will thereby reduce, to a certain extent, the value of “reliability” variable. Importantly, the values of these variables have an impact primarily on the result of solution activity , thus they characterise desired strategy and are taken into account when defining new QoS parameters and settings (QoS settings) with the next iteration of their determination in artificial neural network. The neutral network takes these variables (mode setting) on the inputs of the first layer, and information on the current parameters, bandwidth and use of available connections (observations), and the current QoS parameters and settings (QoS settings) as well as information stored in the internal knowledge base, relating to the archival network and user behaviours. Values of the activation function are calculated in individual neurons of the neural network, giving the specified result for the upper layer of neurons, wherein the type and coefficients of activation function are derived from the best configuration from among 100 most recent iterations, subjected to random modifications of values of individual QoS parameters, each within the range of +/−5%, and the parameters that have no numerical value, such as enabling/disabling prioritization of a certain service, are modified not more than every minute or at a sudden change in the link state (observations). The outputs of the neutral network consist of: knowledge base containing an archive of user activity and network parameters (knowledge) as well as QoS parameters and settings of the network device (QoS settings), QoS parameters and settings of the network device have a direct impact on the quality of services.

By using the invention in embedded system of module of active manageable network device, (mainly due to the implemented artificial neural networks), the embedded system is able to autonomously determine the optical QoS parameters (settings) in order to allow the user to have the temporary optimal QoS parameters set in the device, without the need to manually modify the QoS settings. Modification of parameters and settings is carried out at low level at the embedded system level, not through the intranet interface, thereby eliminating the need to reset the device to operate with the new values of parameters//settings.

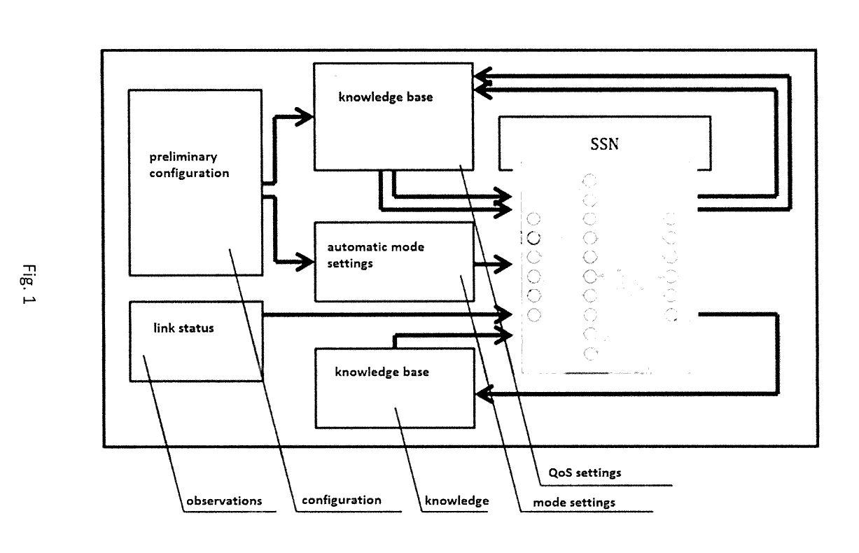

The example of use of method according to the invention is illustrated in the drawing in which FIG. 1 shows a block diagram of this method of network traffic management in information and communication systems.

An example of invention use may be its implementation in an embedded system which constitutes a module of active manageable network device. It is sufficient to supplement the existing device, such as a manageable network switch or router, with an additional, properly designed and manufactured module, consisting of an embedded system with method implemented according to the invention. This module will be able to read and change settings of operating parameters of the network device, collect information on the link status, as well as process the collected data and settings in order to determine the new, optimal QoS parameters and settings. Due to the modular construction of the hardware solution implementing the method according to the invention, the practical implementation of the device may be carried out on the majority of manageable network devices which are available in the market, since these devices are usually microprocessor-based//embedded systems and have the option to read/change values stored in the device memory.

1. A method of network traffic management in information and communication systems, in the mediation network devices for data transmission in information and communication networks, in manageable active network devices which enable the user or administrator to modify settings and parameters relating to QoS via the intranet configuration site of the device or configuration file, and which have mechanisms and settings eliminating or limiting the number of occurrences or the scale of problems specific to information and communication networks, such as: network congestion, latency, jitter, and packet loss, by determining the limits of certain parameters, in particular, the bandwidth, number of connections, session time, as well as by the use of prioritizing, in particular, particular services or connections from/to a specific address, in particular, through a customizable set of policies for configuration of rules and policies capable of reconfiguring the device settings depending on the existing/foreseen in the policy event, wherein, in addition to a configurable set of policies, it has an implemented multilayer artificial neural network which analyses in real time, in discrete points in time, the current parameters of connection and active data transmission sent though the device; based on simple input data comprising, among other things: above data (observations) and the collected data relating to the current activity of users and devices in the network, and archival data concerning their activity, gathered in the internal knowledge base (knowledge), the aforementioned neutral network is able to autonomously and unsupervisedly define and set new values of parameters and settings for QoS (QoS settings) on a managed network device, changing previous parameters and settings without reset, thus allowing trouble-free operation despite the changes of parameters and settings, wherein the user is also able to affect (configuration) the strategy of QoS parameters and settings selection via the intranet site or file with configuration of autonomous mode settings (mode settings); these settings are realised in a clear and comprehensive way for users, using linguistic variables with values in any min-max range, such as: “reliability”, “security” or “efficiency” which affect each other and also the result of solution activity, thus they characterise desired strategy and are taken into account when defining new QoS parameters and settings (QoS settings) with the next iteration of their determination in artificial neural network which takes these variables (mode setting) on the inputs of the first layer, and information on the current parameters, bandwidth and use of available connections (observations), and the current QoS parameters and settings (QoS settings), as well as information stored in the internal knowledge base, relating to the archival network and user behaviours; it calculates values of the activation function in individual neurons, giving the specified result for the upper layer of neurons, wherein the type and coefficients of activation function are derived from the best configuration from among 100 most recent iterations, subjected to random modifications of values of individual QoS parameters, each within the range of +/−5%, and the parameters that have no numerical value, such as enabling/disabling prioritization of a certain service, are modified not more than every minute or at a sudden change in the link state (observations), and the outputs of the neural network consist of: archive of user activity and network parameters (knowledge) as well as QoS parameters and settings of the network device (QoS settings).
